(19) United States
(12) Patent Application Publication
(10) Pub. No.: US 2023/0257769 A1
(43) Pub. Date: Aug. 17, 2023

(54) TRANSGENIC PHOTORECEPTORS AND THEIR USE IN VISION TREATMENT

(71) Applicant: UNIVERSITY HEALTH NETWORK, Toronto (CA)

(72) Inventors: Valerie WALLACE, Toronto (CA); Arturo ORTIN-MARTINEZ, Toronto (CA)

(21) Appl. No.: 18/098,328

(22) Filed: Jan. 18, 2023

Related U.S. Application Data
(60) Provisional application No. 63/300,285, filed on Jan. 18, 2022.

(51) Int. Cl. C12N 15/85; A61K 35/30; A61P 27/02

(52) U.S. Cl. CPC C12N 15/8509; A61K 35/30; A61P 27/02

(57) ABSTRACT

There is described herein a photoreceptor cell comprising a neurotoxin transgene, methods for making the same, and use of the same for vision treatment.

### RELATED APPLICATION

This application claims priority to U.S. Provisional Pat. Application No. 63/300,285 filed on Jan. 18, 2022, the entirety of which is incorporated herein by reference.

### FIELD OF THE INVENTION

The invention relates to transgenic photoreceptors and their use in vision treatment.

### BACKGROUND OF THE INVENTION

It has been reported that transplanted photoreceptors rarely integrate into the recipient retina and instead engage in bidirectional transfer of intracellular materials, including cytosolic GFP and Cre recombinase expressed from transgenes, as well as endogenous phototransduction proteins required for their function in a process termed material transfer (MT) (Decembrini, Martin et al., 2017, Ortin-Martinez, Tsai et al., 2016, Pearson, Gonzalez-Cordero et al., 2016, Santos-Ferreira, Llonch et al., 2016, Singh, Balmer et al., 2016). These observations suggest that cell transplantation-mediated rescue of photoreceptor function is indirect, and at least partially mediated by the transfer of intracellular material to neurons in the recipient retina. MT is facilitated by nanotube-like processes (photoreceptor nanotubes, ^{Ph}NTs) that connect donor and host photoreceptors in vivo (Kalargyrou, Basche et al., 2021, Ortin-Martinez, Yan et al., 2021). The extension of ^{Ph}NTs and MT is regulated by Rho-GTPase remodelling of the actin cytoskeleton such that ^{Ph}NT extension and MT are inhibited when donor cells are induced to express RhoA or a dominant negative Rac1 isoform (Kalargyrou et al., 2021, Ortin-Martinez et al., 2021). To date, however, there are no established approaches to enhance photoreceptor MT.

### BRIEF DESCRIPTION OF THE FIGURES

FIG. 1A shows Schematic of the methodology of retinal transplantation of lentiviral infected photoreceptors. FIG. 1B is a Schematic showing details of the system used to induce TeNT. FIG. 1C is a Validation transgene expression in HEK293T cells transfected with CMV-Empty Vector-P2A-mCherry or CMV-Cre-P2A-mCherry. Cre-recombinase (green) colocalizes with mCherry in the cells transfected with CMV-Cre-P2A-mCherry. FIG. 1D is a Western blot analysis showing GAPDH (green) and Cre-recombinase (red). FIG. 1E is a Confocal microscopy of cryo sections of C57BL/6] recipient retinas 21 days after transplant of ROSATiNT photoreceptors infected with empty vector or Cre lentiviruses. Magnifications of the bolus showing the absence of Vamp2 expression in the cre-expressing donor cells. FIG. 1F is a Quantification of MT in retinas transplanted with control and Cre lentivurses.

FIG. 2 shows the pLM-CMV-R-Cre Addgene #27546 map.

### DETAILED DESCRIPTION

In the following description, numerous specific details are set forth to provide a thorough understanding of the invention. However, it is understood that the invention may be practiced without these specific details.

^{Ph}NTs resemble the basal axon-like process that terminate in synaptic connections to second order neurons in the retina, thus raising the possibility that neurosecretory apparatus might be involved in MT. To investigate the role of neurosecretion in MT we induced expression of tetanus toxin (TeNT) in donor photoreceptors and monitored fluorescence-reporter transfer post transplantation. TeNT blocks neurosecretion by cleaving VAMP2, a component of the SNARE complex that is required for synaptic vesicle docking. We find that TeNT expression in donor cells is associated with a loss of VAMP2 protein, confirming the activity of the toxin, and enhanced fluorescence-reporter transfer to recipient photoreceptors. TeNT expression is the first manipulation that has been shown to enhance MT from transplanted photoreceptors. These results suggest that targeting neurosecretion is an approach to modulate MT of transplanted photoreceptors.

Accordingly, in an aspect, there is provided a photoreceptor cell comprising a neurotoxin transgene.

In some embodiments, the neurotoxin transgene is comprised in a vector transfected into the photoreceptor cell.

In some embodiments, the neurotoxin is tetanus toxin.

In some embodiments, the neurotoxin is botulinum toxin (types A, B, C1, C2, D, E, F or G, preferably types A, B or C).

In some embodiments, the neurotoxin is capable of inhibiting photoreceptor neurosecretion.

In some embodiments, the neurotoxin inhibits at least one protein in the SNARE complex. Preferably, the least one protein in the SNARE complex is VAMP1, VAMP2, Syntaxin or SNAP-25. Preferably, the at least one protein is VAMP2.

In some embodiments, the photoreceptor cell is a human photoreceptor cell.

In a further aspect, there is provided the photoreceptor cell described herein, for use in transplantation into an eye of a subject to restore visual function.

In a further aspect, there is provided a use of the photoreceptor cell described herein, in the preparation of a medicament for transplantation into an eye of a subject to restore visual function. Preferably the use is for the treatment of retinal degeneration and/or damage.

In a further aspect, there is provided a vector comprising a neurotoxin transgene, the neurotoxin capable of inhibiting photoreceptor neurosecretion.

In a further aspect, there is provided the vector described herein, for use in transfecting a photoreceptor cell.

In a further aspect, there is provided a method of preparing a photoreceptor cell for transplantation into a subject, the method comprising transfecting the photoreceptor cell with the vector described herein.

In a further aspect, there is provided a method for treating retinal degeneration and/or damage in an eye of a subject, the method comprising transplanting into the eye the photoreceptor cell described herein.

In a further aspect, the transgenic photoreceptors may be used to induce material transfer and to study if the components of the material transfer lead to regeneration of photoreceptors and as a sensitized model to survey types of transferred cargo, including disease-relevant proteins. For example, the donor photoreceptor (with the toxin transgene) could have knocked-in/knocked-out genes, expressing gene products with expression vectors, involving labelled cellular components (of the material transfer).

Accordingly there is further provided use of the photoreceptor cell described herein in the screening of cellular components of material transfer between photoreceptors. The screening tool would be useful for developing medicament(s) for treatment of eye disorders and/or to restore visual function.

The advantages of the present invention are further illustrated by the following examples. The examples and their particular details set forth herein are presented for illustration only and should not be construed as a limitation on the claims of the present invention.

### Example 1

### Methods and Materials

### Animals and Genotyping

Animal husbandry was in accordance with the Association for Research in Vision and Ophthalmology (ARVO) Statement for the Use of Animals in Ophthalmic and Vision Research. Animals were maintained under standard laboratory conditions and all procedures were performed in conformity with the University Health Network Animal Care Committee (protocol 3499.21) and adhered to the guidelines of the Canadian Council on Animal Care. Adult (6-8 weeks old) C57BL/6J of both sexes were used as transplantation recipients. ROSA^{floxstop-}^{TeNT}mice (Zhang, Narayan et al., 2008) at P0-P3 were used to harvest donor retinal cells.

### Primary Cells Preparation

For retinal dissociates, P0-P3 mice from ROSA^{floxstop-TeNT} (for lentivirus infection, see below) were euthanized by decapitation after a previous exposure of carbon dioxide inhalation for 5 min. Retinas were dissected and collected in CO₂-independent media (18045088, Thermo Fisher Scientific, Mississauga, ON, Canada).

### Tissue Dissociation

Retinal tissues were dissociated to single cells with papain (LK003150, Worthington Biochemical Corp. Lakewood, NJ, USA) according to the manufacturer’s directions. Cells were then washed in Ca²⁺/Mg²⁺-free phosphate buffered saline (PBS) (D8537-500 ML, Merck Millipore Sigma Aldrich, Oakville, ON, Canada), counted using a hemocytometer after staining with a 0.4% Trypan blue viability stain (15250061, Thermo Fisher Scientific, Mississauga, ON, Canada). Cells were then passed through a cell strainer (40 µm) (CLS431750, Thermo Fisher Scientific, Mississauga, ON, Canada) before being re-suspended in retinal explant medium supplemented with SATOs for cell culture.

### Lentiviral Infection

Retinal dissociates from P0-3 mice were resuspended in REM and plated at a concentration of 3×10⁷ cells in 10 ml of REM into 10 cm Primaria™ dishes (353801, BD Falcon, BD Biosciences, Oakville, ON, Canada). Lentiviruses were added to the plate at a titer to achieve >80% infection efficiency. All cultures were maintained in 5% CO₂-buffered incubators (50116048, Heracell™ 150i CO₂, Thermo Fisher Scientific, Asheville, NC, USA) at 37° C. After 2 days in vitro (DIV), cells were de-adhered with Trypsin-EDTA (0.05% Trypsin with EDTA 4Na) (25300062, Thermo Fisher Scientific, Mississauga, ON, Canada) for 5 minutes and the enzymatic digestion was stopped with 10% BSA. Cells were washed in Ca²⁺/Mg²⁺ -free PBS 4 times and filtered through a 40 µm cell strainer. Cells were resuspended at a concentration of 200,000 cells per µl in 0.005% DNAse in EBSS and kept on ice prior to transplantation or cell culture.

### Lentivirus Design, Cloning and Production

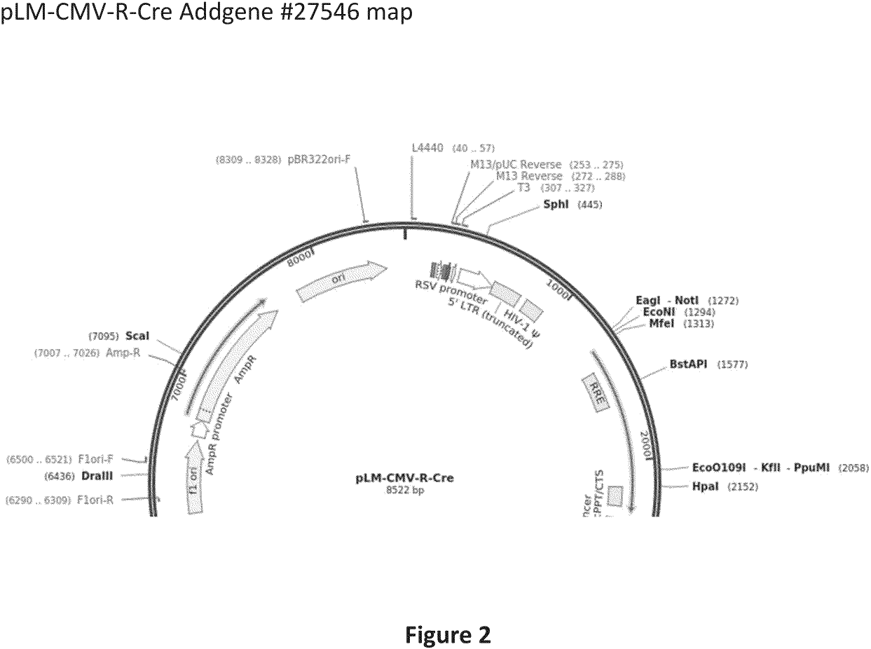

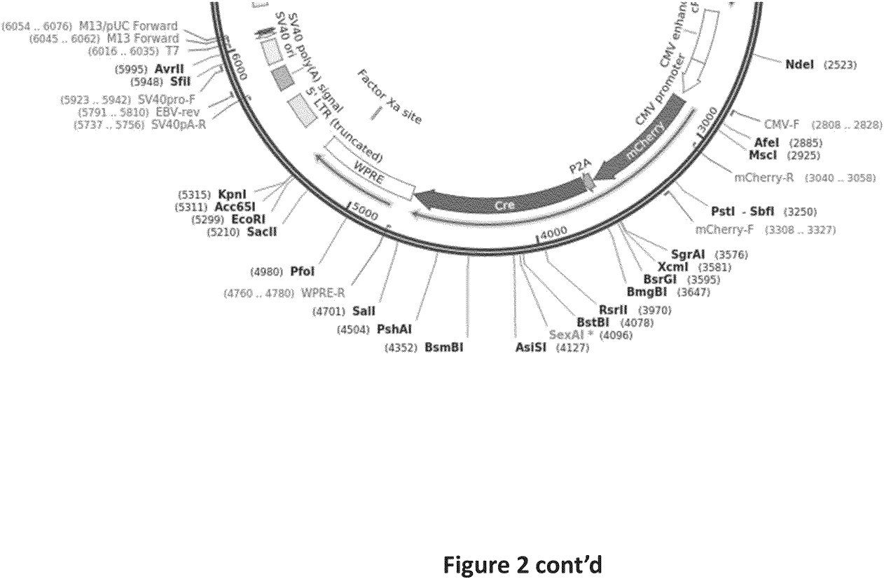

Cre cDNAs was remove after restriction enzymes digestion from the pLenti-CMV-mCherry-P2A-Cre vector backbone (derived from pLM-CMV-R-Cre Addgene #27546). The constructs were verified by Western blot. Lentiviruses were produced by Lipofectamine3000 (L3000015, Invitrogen Canada Inc. Burlington, ON, Canada) mediated transfection of transfer plasmid with 2^{nd} generation lentiviral packaging system (pMD2.G and psPAX2) into 293FT cells (ATCC; CRL-1573™). Supernatant was collected 48 h post-transfection and concentrated by centrifugation at 100,000 x g for 3 h. The lentivirus was resuspended at approximately 1/150 of its original volume in either PBS (D8662-500ML, Merck Millipore Sigma Aldrich, Oakville, ON, Canada) or DMEM (D5796-500ML, Merck Millipore Sigma Aldrich, Oakville, ON, Canada), then frozen at -80° C. Virus titer was determined by the frequency of RFP expression in HEK293T cells. See FIG. 2.

### Subretinal Injection

Dissociated cells were resuspended at a concentration of 200,000 cells per µl in 0.005% DNAse in EBSS and kept on ice prior to transplantation. Adult recipient C57BL/6J mice (6-8 weeks old) were anesthetized using a mixture of ketamine (100 mg/ml, Ketalean) (8KET004D, Bimeda MTC Animal Health Inc. Cambridge, ON, Canada) at 50 mg/kg and medetomidine (1 mg/ml, Cepetor) (236 1506 0, Modern Veterinary Therapeutics LLC, Miami, FL, USA) at 1 mg/kg in sterile 0.9% NaCl (JB1300, Baxter Corp. Mississauga, ON, Canada) administered intraperitoneally. Pupils were dilated using 1% tropicamide (Mydriacyl) (0065-0355-03 Alcon, Mississauga, ON, Canada) drops, followed by application of a 0.2% hydromellose gel (Genteal Tears) (0078-0429-57, Alcon, Mississauga, ON, Canada) to maintain proper eye lubrication. For injections the left eye was gently prolapsed and then immobilized using a customized latex dam to allow free blood circulation. The retina fundus was visualized under a dissection microscope, adjusting the reflexion index, for confirmation of cell deposit. A microscopy-guided microinjection was performed by a second surgeon located orthogonal to the viewing microscope. A scleral incision was made in the dorsal side, posterior to the limbus using a 30-gauge needle (305136, VWR International, LLC. Mississauga, ON, Canada). Next, a blunt 32-gauge needle (7762-05, Hamilton Company, Montreal, QC, Canada) was inserted tangentially into the subretinal space (SRS) and advanced in the SRS under the guidance of the person visualizing the fundus. Once the needle was located in the SRS, a small incision was made in the cornea to relieve intraocular pressure. 1.0 µl of cell suspension (200,000 donor cells) was injected over 30 seconds using a Remote Infuse/Withdraw Pump 11 Elite Nanomite Programmable Syringe Pump (70-4507, Harvard Apparatus, Saint Laurent, QC, Canada) connected to a Compact Mouse and Rat Stereotaxic Instrument, Dual Manipulator (75-1827, Harvard Apparatus, Saint Laurent, QC, Canada). After a 30-second pause to allow for equilibration of pressure and to prevent reflux, the needle was then slowly retracted, and the animal anaesthesia reversed using an intraperitoneal injection of 1 mg/kg atipamezole (5 mg/ml Revertor) (236 1504 0, Modern Veterinary Therapeutics LLC, Miami, FL, USA). Animals were placed on a heating pad and monitored until fully recovered.

### Tissue Processing

Recipient mice were euthanized by carbon dioxide inhalation and transcardially perfused with PBS for exsanguination and 4% PFA for fixation. Eyes were then marked with a silver nitrate stick (118-395, AMG Medical Inc. Mont-Royal, QC, Canada) on the dorsal region while maintaining the caruncle as an anatomical spatial reference. For cryosectioning, tissues were fixed for an additional 30 minutes in 4% PFA on ice and then cryoprotected overnight in 30% sucrose (SUC507.1, BioShop Canada Inc. Burlington, ON, Canada) PBS solution at 4° C. Tissues were equilibrated in 50:50 30% sucrose in PBS:OCT (Tissue-Tek®) (4583, Sakura Finetek USA Inc. Maumee, OH, USA) for 1 hour and then oriented and embedded in plastic molds. Tissue blocks were stored at -80° C. Tissue was sectioned at 20 µm thickness onto Superfrost Plus slides (12-550-15 Thermo Fisher Scientific, Mississauga, ON, Canada) on a cryostat (CM3050 S, Leica Biosystems, Leica Microsystems Canada Inc. Richmond Hill, ON, Canada) and air-dried for 1 hour before being stored in a slide box with desiccant at -20° C.

### Immunohistochemistry (IHC)

Retina cyrosections were permeabilized with PBS 0.5% Tx then blocked with 2% DS 0.5% Tx in PBS. Slides were incubated with primary antibodies for RFP (which detects mCherry) and Vamp2 diluted in 2% DS 0.5% Tx in PBS overnight at 4° C. in a light protected humidified box. After three washes with PBS, sections were incubated with fluorescent secondary antibodies diluted in 0.5% Tx in PBS for 2 hours at RT and nuclei were counterstained with Hoechst 33342 (62249, Thermo Fisher Scientific, Mississauga, ON, Canada). Slides were washed and glass coverslips were mounted with DAKO mounting media (S3023, Cedarlane, Burlington, ON, Canada).

### Immunoblotting

Total protein extracts were prepared from HEK290 cell cultures by manual homogenization in ice-cold RIPA lysis buffer (50 mM Tris-HCl (10812846001, Merck Millipore Sigma Aldrich, Oakville, ON, Canada) pH 7.4, 150 mM NaCl (567440, Merck Millipore Sigma Aldrich, Oakville, ON, Canada), 1% NP40 (492016, Merck Millipore Sigma Aldrich, Oakville, ON, Canada), 0.1% SDS (428018-200ML, Merck Millipore Sigma Aldrich, Oakville, ON, Canada) 0.5% sodium deoxycholate (30970, Merck Millipore Sigma Aldrich, Oakville, ON, Canada), 10 mM NaF (S7920-100G, Merck Millipore Sigma Aldrich, Oakville, ON, Canada), 5 mM sodium citrate (80552, Merck Millipore Sigma Aldrich, Oakville, ON, Canada) 1.5 mM MgCl₂ (M8266-100 G, Merck Millipore Sigma Aldrich, Oakville, ON, Canada), 10 µM ZnCl₂ (39059-1ML-F, Merck Millipore Sigma Aldrich, Oakville, ON, Canada)) supplemented with a protease inhibitor cocktail (11836170001, Roche, Merck Millipore Sigma Aldrich, Oakville, ON, Canada) and phosphatase inhibitor cocktail (5870S, Cell Signaling Technologies, Danvers, MA, USA). Homogenates were sonicated for 10 s at 4° C., followed by centrifugation at 15,000 g for 20 min at 4° C. to sediment insoluble material. Protein concentration was determined with Bradford reagent (B6916-500 ML, Merck Millipore Sigma Aldrich, Oakville, ON, Canada), and samples were resolved by denaturing SDS-PAGE on 4-20% Tris-acetate polyacrylamide gels (4561094, Bio-Rad Laboratories (Canada) Ltd. Mississauga, ON, Canada), and blotted onto PVDF membranes (1620177, Bio-Rad Laboratories (Canada) Ltd. Mississauga, ON, Canada) by electrophoretic transfer in Tris-glycine buffer containing 10% methanol (6701-7-40, Caledon Laboratories Ltd. Georgetown, ON, Canada) and 0.05% SDS. Membranes were blocked with 5% BSA in Tris-buffered saline (TBS), followed by an incubation for 1 h at RT and overnight at 4° C. with primary antibodies for RFP and Cre, and a subsequent incubation for 2 h at RT with secondary antibodies. Membranes were subjected to three 10 min washes in TBS following primary and secondary antibody incubation.

### Quantification of Material Transfer Index

The number of RFP⁺ cell bodies in the recipient ONL and the number of RFP⁺ cells in the adjacent subretinal space were counted. The total number of RFP⁺ cells per eye was determined by extrapolation, based on quantification of every 4^{th} section. We previously observed, by way of Pearson coefficient and Linear regression analyses, a strong correlation between the number of RFP⁺ cells in the recipient ONL (corresponding to recipient cells that received transferred RFP) and the number of RFP⁺ cells in the SRS. Thus, we normalized the number of RFP⁺ cells in the ONL to the number of RFP⁺ cells in the SRS and refer to this parameter as material transfer index.

### Confocal Imaging

Cryostat tissue sections were imaged using an LSM 780 (Carl Zeiss Inc. Thornwood, NY, USA) confocal microscope. Images were acquired at a minimum of 2048 × 2048 pixels resolution at a maximum of 5% laser intensity and a maximum 800 digital gain. 20x objective was used at a 1.0 Airy Unit pinhole size. Comparative images were taken using identical acquisition parameters.

### Results and Discussion

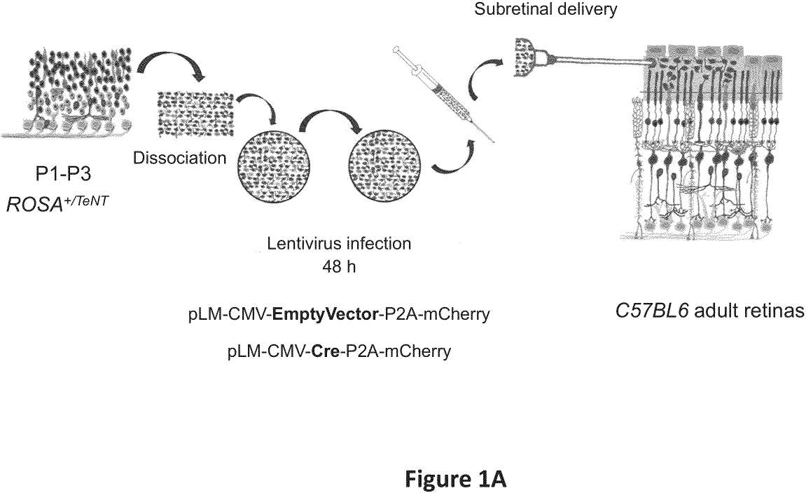

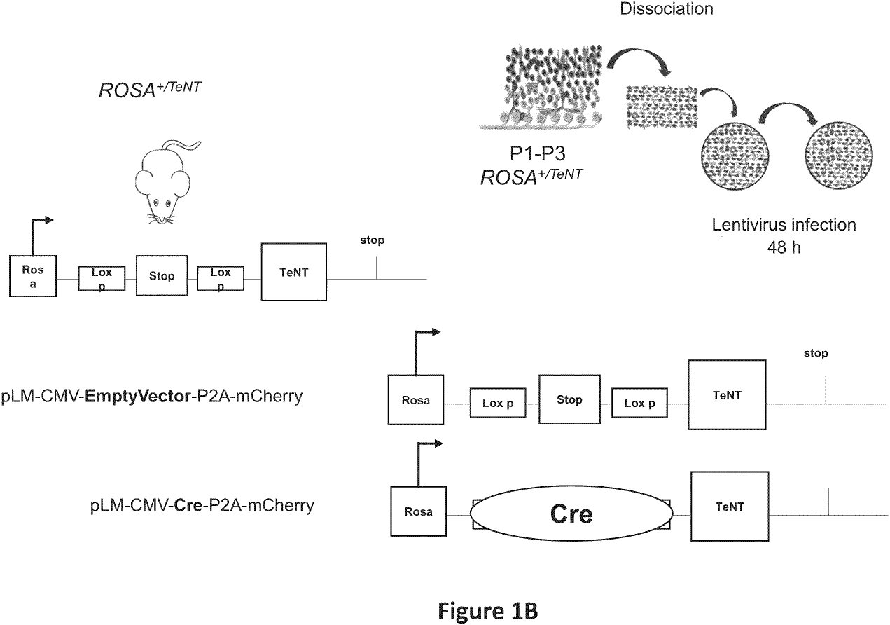

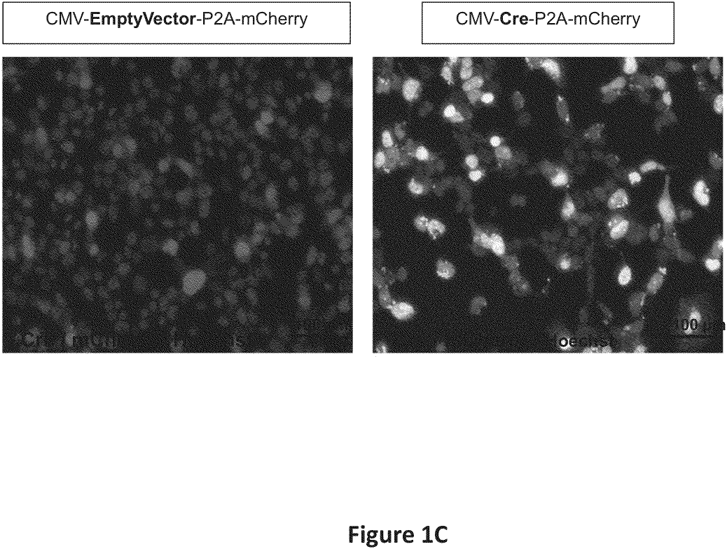

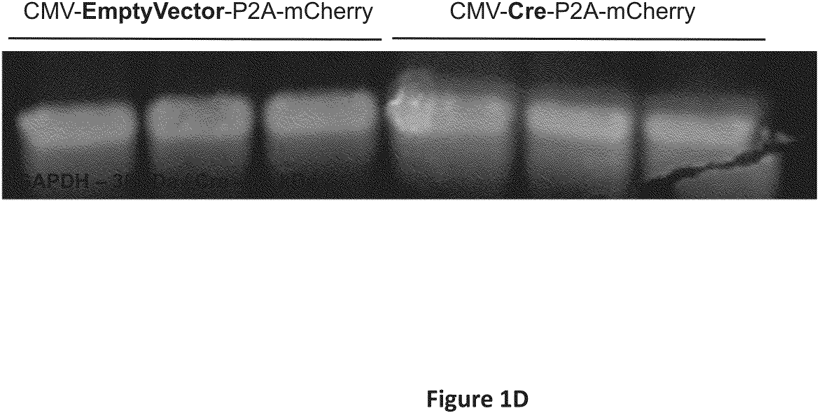

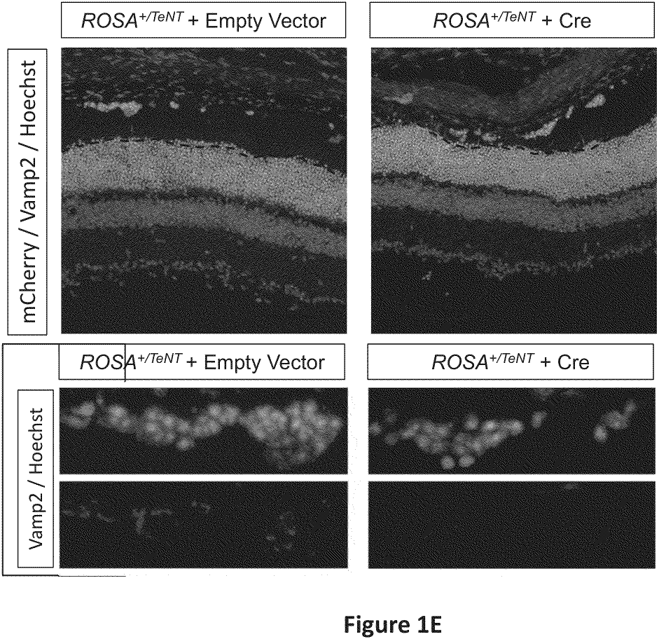

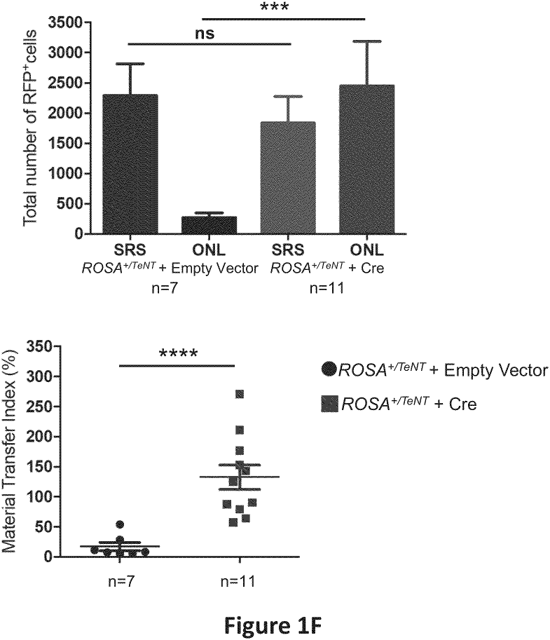

To investigate the effect of TeNT on MT we used photoreceptors from P0-P3 ROSA^{floxstop-TeNT} mice, which harbour a Cre-dependent Tetanus toxin light chain transgene knocked into the ROSA locus (Zhang, Narayan et al., 2008) (FIG. 1A). Retinal dissociates were infected with lentiviruses expressing mCherry (Lenti-CMV- Empty vector-P2A-mCherry; control) and Cre-mCherry (CMV-Cre-P2A-mCherry, Cre) and photoreceptors were transplanted to the subretinal space (SRS) of wildtype adult retinas [FIGS. 1B-D]. Induction of the TeNT transgene in donor cells was confirmed by reduced VAMP2 staining specifically in Lenti-Cre-infected donor cells in the SRS [FIG. 1E] and MT was monitored 21 days after transplantation by IHC for mCherry, which is expressed by the donor cells. MT of fluorescent reporter proteins to host photoreceptors correlates with the number of surviving donor cells in the SRS (Ortin-Martinez et al., 2021), thus we expressed the MT index as the ratio of mCherry+ host/mCherry+ donor cells. Compared with control lentivirus-infected cells, retinas transplanted with TeNT+ donor cells exhibited a significant increase in MT (MT index control: 17.32±6.86, TeNT: 132.73±20.19) (FIG. 1F). The enhanced MT associated with TeNT induction was not associated with a change in donor photoreceptor survival in the SRS (control: 2291± 529 (n=7) versus TeNT: 1835±441 (n=11)) (FIG. 1F). We therefore conclude that TeNT expression in donor photoreceptors is sufficient to reduce VAMP2 levels and to enhance MT to host photoreceptors.

### Example 2

### Methods and Materials

### Modulate GNAT1 Transfer to Recipient Photoreceptors in a Mouse Model of GNAT1 Deficiency

We will use our well-established workflow for lentivirus infection and transplantation of perinatal mouse photoreceptors to deliver GFP-tagged donor cells to the subretinal space in adult mice deficient for GNAT1. To inhibit MT, we will transplant donor cells infected with lenti-RhoA-GFP and to enhance MT we will transplant donor cells infected with lenti-TeNT-GFP. After 3 weeks, retinas will be harvested and the transfer index for GNAT1 and GFP (number of host cells with transferred protein normalized to the number of donor cells in the subretinal space) will be determined, as we have done previously. To directly test the relationship between GNAT1 expression and the level of transfer we will also compare transfer in retinas transplanted with GNAT1 overexpressing photoreceptors.

Retinas from ROSA-TeNT (a cre inducible form of Tetanus toxin) will be dissected from postnatal day 0 mice, dissociated to single cell suspensions with papain and plated in tissue culture dishes. Cells will be incubated with lentiviruses (empty, Cre recombinase, RhoA, all with a GFP reporter protein) and 24 hours later the cells will be collected, washed and resuspended to a final concentration of 200,000 cells/microlitre. Adult recipient GNAT1KO mice will be anesthetized, pupils dilated and injected with 1 microlitre of cell suspension (200,000 cells) to the subretinal space. At least 6 recipients/group will be injected. 3 weeks after surgery the retinas will be harvested and stained for GFP and GNAT1 (to track donor cells and host cells with transferred GFP and GNAT1) and the numbers of surviving donor cells and host cells with GFP and GNAT1 will be counted. The degree of MT is expressed at the # host cells with transferred protein/ number of surviving donor cells in the subretinal space.

### Investigate the Requirement for MT for Restoring Light-Dependent Behavior and Neuronal Responses in Blind Mice

We predict that GNAT1 transfer to GNAT1 KO photoreceptors will restore light-dependent GC responses, the primary output of the retina. We will test mice transplanted with GFP, RhoA-GFP and TeNT-GFP expressing donor cells in a light aversion assay (mice with photoreceptor function will avoid the illuminated side of the chamber) adapted for rod function. We will also record light-activated spiking activity from GCs using single and field recording of transplanted retinas and correlate changes in GC activity the position of transplanted donor cells, which will be visible as GFP signal. As a control to confirm that the light responsiveness of transplanted retinas is a function of host photoreceptors and not light responsiveness from the donor cells, we will transplant retinas with donor cells from rhodopsin KO mice, which lack all rod function but still express GNAT1. To degree of MT-associated rescue will be benchmarked against retinas transduced with AAV-GNAT1 viral vectors. To obtain statistically meaningful data we will record from 6 mice/group at 3 weeks post transplantation.

### Results and Discussion

Photoreceptor transplantation improves vision in mouse models of IRD, a rescue that was reported to be mediated by the physical integration of donor cells into the host synaptic circuitry. However, we describe above that donor cells do not physically integrate into the host retina, but instead exchange intracellular proteins with recipient photoreceptors in a process termed material transfer (MT). Salient to this example, proteins that are affected in IRDs can be transferred, raising the possibility that sufficient amounts of protein can be transferred to restore photoreceptor function.

Nanotube formation and material transfer efficiency requires RhoGTPase actin remodeling and can be inhibited by transgenic expression of RhoA and dominant negative Rac1. As described above, inhibiting neurosecretion by inducing the expression of a tetanus toxin transgene in photoreceptors enhances MT in transplanted retinas in vivo. Thus, we have generated tools available to modulate MT for therapeutic use. Photoreceptor transplantation represents a gene agnostic therapeutic approach to restore photoreceptor function, since transplanted cells could potentially transfer proteins and organelles that are present at inadequate levels or in mutant form in genetically deficient photoreceptors. Importantly, exploiting MT would not require disease specific manipulations to donor cells, which would expand the patient pool that could be treated. Here we investigate the requirement for MT to promote the accumulation of GNAT1 and restoration of function in GNAT1 deficient rod photoreceptors in vivo.

From the methods above, we expect to detect GNAT1 transfer to host photoreceptors and for this to be suppressed or enhanced by RhoA and TeNT expression in donor cells, respectively. We do not foresee a problem with this as we and other have documented GNAT1 transfer to mutant photoreceptors. There is also some evidence that GNAT1 transfer can be inhibited with ectopic RhoA expression in donor cells.

Further, we expect a reduction in rescue in retinas transplanted with RhoA expressing photoreceptors relative to wildtype and an enhanced rescue in retinas transplanted with TeNT-expressing donor cells.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

Decembrini S, Martin C, Sennlaub F, Chemtob S, Biel M, Samardzija M, Moulin A, Behar-Cohen F, Arsenijevic Y (2017) Cone Genesis Tracing by the Chrnb4-EGFP Mouse Line: Evidences of Cellular Material Fusion after Cone Precursor Transplantation. Molecular therapy : the journal of the American Society of Gene Therapy 25: 634-653
Kalargyrou AA, Basche M, Hare A, West EL, Smith AJ, Ali RR, Pearson RA (2021) Nanotube-like processes facilitate material transfer between photoreceptors. EMBO Rep 22: e53732
Ortin-Martinez A, Tsai EL, Nickerson PE, Bergeret M, Lu Y, Smiley S, Comanita L, Wallace VA (2016) A Reinterpretation of Cell Transplantation: GFP Transfer from Donor to Host Photoreceptors. Stem cells (Dayton, Ohio)
Ortin-Martinez A, Yan NE, Tsai ELS, Comanita L, Gurdita A, Tachibana N, Liu ZC, Lu S, Dolati P, Pokrajac NT et al. (2021) Photoreceptor nanotubes mediate the in vivo exchange of intracellular material. The EMBO journal 40: e107264
Pearson RA, Gonzalez-Cordero A, West EL, Ribeiro JR, Aghaizu N, Goh D, Sampson RD, Georgiadis A, Waldron PV, Duran Y et al. (2016) Donor and host photoreceptors engage in material transfer following transplantation of post-mitotic photoreceptor precursors. Nature communications 7: 13029
Santos-Ferreira T, Llonch S, Borsch O, Postel K, Haas J, Ader M (2016) Retinal transplantation of photoreceptors results in donor-host cytoplasmic exchange. Nature communications 7: 13028
Singh MS, Balmer J, Barnard AR, Aslam SA, Moralli D, Green CM, Barnea-Cramer A, Duncan I, MacLaren RE (2016) Transplanted photoreceptor precursors transfer proteins to host photoreceptors by a mechanism of cytoplasmic fusion. Nature communications 7: 13537
Zhang Y, Narayan S, Geiman E, Lanuza GM, Velasquez T, Shanks B, Akay T, Dyck J, Pearson K, Gosgnach S et al. (2008) V3 spinal neurons establish a robust and balanced locomotor rhythm during walking. Neuron 60: 84-96

1. A photoreceptor cell comprising a neurotoxin transgene.

2. The photoreceptor cell of claim 1, wherein the neurotoxin transgene is comprised in a vector transfected into the photoreceptor cell.

3. The photoreceptor cell of claim 1, wherein the neurotoxin is tetanus toxin.

4. The photoreceptor cell of claim 1, wherein the neurotoxin is botulinum toxin (types A, B, C1, C2, D, E, F or G, preferably types A, B or C).

5. The photoreceptor cell of claim 1, wherein the neurotoxin is capable of inhibiting photoreceptor neurosecretion.

6. The photoreceptor cell of claim 5, wherein the neurotoxin inhibits at least one protein in the SNARE complex.

7. The photoreceptor cell of claim 6, wherein the least one protein in the SNARE complex is VAMP1, VAMP2, Syntaxin or SNAP-25.

8. The photoreceptor cell of claim 7, wherein the at least one protein is VAMP2.

9. The photoreceptor cell of claim 1, wherein the photoreceptor cell is a human photoreceptor cell.

10. The photoreceptor cell of claim 1, for use in transplantation into an eye of a subject to restore visual function.

11. A vector comprising a neurotoxin transgene, the neurotoxin capable of inhibiting photoreceptor neurosecretion.

12. The vector of claim 11, for use in transfecting a photoreceptor cell.

13. A method of preparing a photoreceptor cell for transplantation into a subject, the method comprising transfecting the photoreceptor cell with the vector of claim 11.

14. A method for treating retinal degeneration and/or damage in an eye of a subject, the method comprising transplanting into the eye the photoreceptor cell of claim 1.

15. A method for identifying cellular components of material transfer between photoreceptors to identify a medicament for eye treatment, the method comprising screening using the photoreceptor cell of claim 1.
